(19) United States
(12) Patent Application Publication
(10) Pub. No.: US 2008/0138909 A1
(43) Pub. Date: Jun. 12, 2008

(54) Mass Spectrometry of Arginine-Containing Peptides

(75) Inventors: Susan Wheeler, Oxford (GB); Mikhail Sergeevich Shchepinov, Oxford (GB); Edwin Mellor Southern, Oxford (GB)

Correspondence Address: WENDEROTH, LIND & PONACK, L.L.P., 2033 K STREET N. W., SUITE 800, WASHINGTON, DC, 20006-1021, US

(73) Assignee: OXFORD GENE TECHNOLOGY IP LIMITED, Yarnton Oxford (GB)

(21) Appl. No.: 10/581,683

(22) PCT Filed: Dec. 8, 2004
(86) PCT No.: PCT/GB2004/005210

§ 371 Date: Aug. 29, 2007

(30) Foreign Application Priority Data
Dec. 8, 2003 (GB) 0328414.8
Dec. 8, 2003 (GB) 0328417.1
Sep. 22, 2004 (EP) 04104605.3

(51) Int. Cl. G01N 33/00; C07K 7/00; G06G 7/48

(52) U.S. Cl. 436/89; 436/86; 530/300; 530/330; 703/11

(57) ABSTRACT

Peptides can be derivatised such that, when ionised and analysed by mass spectrometry, those containing arginine residues give characteristic peak patterns. Peaks corresponding to arginine-containing peptides can therefore be selected from a mass spectrum in order to simplify and improve peptide analysis. Suitable labels give derivatised peptides that have the ability to form both a stabilised ion species ([P]+) and a protonated ion molecular species ([P+H]+) that differ by one average mass unit. A characteristic peak pattern is seen.

### TECHNICAL FIELD

This invention relates to methods of analysing peptides by mass spectrometry. The invention further relates to peptides useful in the methods of the invention.

### BACKGROUND OF THE INVENTION

“Peptide mass fingerprinting” (also known as peptide mass mapping) is an inexpensive, sensitive, accurate, high-throughput and user-friendly method for the identification of a protein of interest. The protein of interest is identified via an analysis of the mass spectrum of the peptides formed by enzymatic digestion of the protein. The “peptide mass fingerprint” derived from this mass spectrum is a list of peptide mass values for the peptides of the protein digest and is used to identify the protein by database searching. Unambiguous identification of the protein by database searching can be achieved where the peptide mass fingerprint contains a minimum number of peptides per protein with a unique combination of monoisotopic masses.

In a typical peptide mass fingerprinting protocol, the protein of interest is initially separated from other proteins. The initial separation step may be based upon any of a number of protein characteristics (such as isoelectric point, molecular weight, charge or hydrophobicity) and may be achieved by a variety of methods (such as two-dimensional polyacrylamide gel electrophoresis).

The protein of interest is then digested with a protease enzyme to produce a mixture of peptides. For example, following separation by two-dimensional polyacrylamide gel electrophoresis, individual spots can be digested with a specific protease. A number of different proteases are commonly used in peptide mass fingerprinting.

The mass spectrum of the mixture of protein digest is then obtained by mass spectrometry. Typically, MALDI-MS is used.

The peptide mass fingerprint is subsequently used to search databases to identify the protein of interest. The peptide monoisotopic masses are compared with the expected monoisotopic masses in the databases. A number of algorithms are known which attempt to identify the protein of interest from the peptide mass fingerprint. The most commonly used are MASCOT, ProFound, MSFit, PROWL and SEQUEST.

Although peptide mass fingerprinting allows direct identification of the protein of interest from the peptide mass fingerprint, there are a number of factors that can reduce the efficiency of this method as a tool for protein identification. For example, a large number of false positive matches may be returned after database searching. Particularly, it is difficult to distinguish proteins that give rise to highly similar peptide mass fingerprints.

As a result, it is often not possible to unambiguously identify the protein of interest by peptide mass fingerprinting. This leads to the need for additional time-consuming and expensive mass spectrometry protocols. There is thus a need for improvements in peptide mass fingerprinting that increase the likelihood of unambiguous protein identification.

Among the thousands of proteins expressed in a typical mammalian cell, as many as one third are now thought to be phosphorylated. A knowledge of the particular residues that are phosphorylated on a protein can provide an insight into signalling pathways, by permitting an understanding the regulation of that protein's activity. However, isolating and sequencing phosphopeptides derived from protein digests to identify specific phosphorylated residues remains a labour intensive, time-consuming challenge. Mass spectrometry has been used for detecting the presence of post-translational modifications or modified amino acids and locating the position of the specific modified residues. To date, the most successful method used is electrospray tandem mass spectrometry via neutral loss scanning within the negative ion mode for the identification of phosphopeptide-specific marker ions from high energy CID fragmentation followed by low energy CID to determine amino acid sequence from the often in-source de-phosphorylated peptide. In contrast, during MALDI-TOF-MS analysis phosphopeptides can remain undetected because of poor ionisation efficiency, low site occupancy and metastable ion formation through fragmentation; particularly peptides containing phosphorylated serine and threonine residues. In such cases, phosphopeptides need to be enriched by IMAC chromatography prior to mass spectrometry analysis with ‘cooler’ matrices. However, there remains a need for further and improved methods to identify post-translationally-modified peptides, and in particular phosphopeptides, within a peptide mixture and to allow determination of the proportion of unmodified to modified peptide.

### DISCLOSURE OF THE INVENTION

The inventors have now found that peptides can be derivatised such that, when ionised and analysed by mass spectrometry, those containing arginine residues give characteristic peak patterns. Peaks corresponding to arginine-containing peptides can therefore be selected from a mass spectrum in order to simplify and improve peptide analysis. Suitable labels give derivatised peptides that have the ability to form both a stabilised ion species ([P]⁺) and a protonated ion molecular species ([P+H]⁺) that differ by one average mass unit. Because derivatised arginine-containing peptides can form these two different species, a characteristic peak pattern is seen for those peptides. The stabilised ion species ([P]⁺) may be less abundant than the protonated ion molecular species ([P+H]⁺) but may also be more abundant or equally abundant. This peak pattern (e.g. see FIG. 2) is not seen for derivatised peptides that do not contain arginine residues.

The invention provides a method of analysing a peptide by mass spectrometry, comprising the steps:
a) reacting the peptide with a label to provide a derivatised peptide that, if the peptide contains an arginine residue, can form both a stabilised ion species ([P]⁺) and a protonated ion molecular species ([P+H]⁺) that differ by one average mass unit; and
b) analysing the derivatised peptide by mass spectrometry to provide a mass spectrum.

The method will generally include the further step of:
c) analysing the mass spectrum to determine if it contains a peak pattern for a peptide in which a first monoisotopic mass peak and a second monoisotopic mass peak are separated by one average mass unit.

The first peak in the spectrum analysed in step (c) may be (i) less abundant than the second peak, and (ii) of lower mass than the second peak.

Whereas mass spectrometrical methods for the identification of peptides that contain arginine residues are known in the prior art [Leitner & Lindner (2003) Journal of Mass Spectrometry 38:891-899], these prior art methods involve a characteristic mass shift and require a comparison of derivatised and underivatized samples to determine the presence or absence of the derivatised arginine residues. In contrast, the methods of the invention give additional information in the form of a characteristic peak pattern rather than a characteristic mass shift (although the label does, of course, increase a peptide's mass). Whereas the prior art methods require at least two mass spectrometry data sets (underivatized and derivatised) to identify those peptides that contain arginine residues, therefore, the invention requires only a single (derivatised) mass spectrometry data set, thereby allowing greatly simplified identification. Comparison of two data sets is not, however, excluded.

Without wishing to be bound by any theory, it is believed that the characteristic peak pattern observed for derivatised arginine-containing peptides results from the detection of singly-charged ionic free radical forms of the derivatised peptides. The detection of singly charged ionic free radical forms of the derivatised peptides is believed to be possible due to the presence of an arginine side chain (—(CH₂)₃)—NH—C(NH)NH₂) in conjunction with stabilisation of the free radical by the label. The same characteristic peak pattern is also observed for peptides that contain homoarginine, which is included within the scope of the term arginine herein.

The invention also provides a method of analysing a peptide mass spectrum, comprising the step of analysing the spectrum to determine if it contains a peak pattern for a peptide in which a first peak and a second peak are separated by one average mass unit. The first peak may be less abundant than the second peak. The spectrum will typically be a deisotoped spectrum, and will also typically be a centroided spectrum.

The invention also provides a method of identifying a protein by mass spectrometry, comprising the steps:
a) obtaining a mass spectrum of a mixture of peptides derived from a protein, wherein the peptides carry a label such that, if the peptide contains an arginine residue, it can form both a stabilised ion species ([P]⁺) and a protonated ion molecular species ([P+H]⁺) that differ by one average mass unit;
b) analysing the mass spectrum to identify if, after optional deisotoping, it contains a peak pattern for a peptide in which a first peak and a second peak are separated by one average mass unit; and
c) searching a database using information generated in step b) to identify the protein.

The first peak in the spectrum analysed in step (b) may be (i) less abundant than the second peak, and (ii) of lower mass than the second peak.

Within step b), the method will generally involve analysing the spectrum to identify monoisotopic masses of the peptides, and step c) will use this peptide monoisotopic mass information.

### The Sample

The peptide analysed by the methods of the invention will be within a sample, and that sample may comprise a single peptide or a mixture of different peptides.

The term “peptide” includes any molecule comprising two or more amino acids joined to each other by peptide bonds or modified peptide bonds, i.e. peptide isosteres. This term refers both to short chains (e.g. oligopeptides with fewer than 20 amino acids) and to longer chains (e.g. polypeptides with 20 or more amino acids).

The peptide may be a linear, cyclic or branched peptide. The peptide should have a free N-terminus. Preferably, the peptide is a linear peptide.

The peptides may contain either L- and/or D-amino acids. Preferably, the peptides contain L-amino acids only (including glycine).

The peptides may contain amino acids other than the 20 ‘classical’ gene-encoded amino acids. For example, the peptides may contain amino acids incorporated directly by an unusual mRNA translation step (e.g. selenocysteine). The peptides may also contain amino acids produced by metabolic conversions of free amino acids (e.g. ornithine and citrulline). The peptides may also contain amino acids that include post-transitional modifications (e.g. acetylation, amidation, deamidation, biotinylation, C-mannosylation, flavinylation, farnesylation, formylation, geranyl-geranylation, lipidation, phosphorylation, glycosylation, hydroxylation, disulphide bond formation, methylation, myristoylation, sulphation, carboxylation, ADP-ribosylation, etc.). The peptides may also contain amino acids that have been modified by chemical modification techniques, which are well known in the art.

The methods of the invention are directly applicable to the analysis of post-translationally modified peptides, in particular phosphopeptides, as illustrated by the Examples herein. A sample may contain a peptide in both modified and unmodified form.

The modifications that occur in a peptide often will be a function of how the peptide is made. For peptides that are made recombinantly, the nature and extent of the modifications in large part will be determined by the post-translational modification capacity of the particular host cell and the modification signals that are present in the amino acid sequence of the peptide in question. For instance, glycosylation patterns vary between different types of host cell.

Modifications can occur anywhere in the peptide, including the peptide backbone, the amino acid side-chains and the amino or carboxyl termini. Blockage of the amino or carboxyl terminus in a peptide by a covalent modification is common in naturally-occurring and synthetic polypeptides and such modifications may be present in the peptides.

The peptides can be prepared in any suitable manner. For example, the peptides may be prepared biologically (for example, by culture of naturally-occurring or recombinant cell types), or may be prepared synthetically (for example, by chemical synthesis).

A mixture of peptides includes 2 or more different peptides, e.g. >5 peptides, >10 peptides, >20 peptides, >30 peptides, >40 peptides, >50 peptides, >60 peptides, >70, peptides, >80 peptides, >90 peptides, >100 peptides, etc. Peptide mixtures can be prepared in any suitable manner. For example, the mixture of peptides may be prepared directly from a cell type of interest (its proteome in whole or part), or may be prepared by cleavage of one or more polypeptides. Polypeptide cleavage may be enzymatic or non-enzymatic. Suitable enzymatic reagents include, but are not limited to, Trypsin, Arg-C, Asp-N, Asp-N-ambic, chymotrypsin, Lys-C, Lys-C/P, PepsinA, S. Aureus pH 4, S. Aureus pH 8, Pancreatic Elastase, Thermolysin, Clostripain, V8-DE, V8-E, Thrombin, Factor Xa Protease, Enterokinase, endopeptidase rTEV from tobacco etch virus, 3C human rhinovirus protease, etc. Suitable non-enzymatic cleavage reagents include, but are not limited to, CNBr, Formic acid, Hydroxylamine, Hydroxylamine, etc.

Preferably, a mixture of peptides is prepared by digesting one or more proteins with a protease. The protease enzyme may be any suitable protease enzyme. Preferably, the protease enzyme is selected for its cleavage specificity. Enzymes that cleave proteins indiscriminately will lead to a mixture of peptides producing a complex mass spectrum. Conversely, enzymes that cleave only at very rare positions will lead to a mixture of peptides producing a simple mass spectrum from which it may not be possible to unambiguously identify the protein. Examples of commonly used protease enzymes are given in the previous paragraph.

Preferred proteases are those that can cleave the protein to produce peptides that comprise an N-terminal and/or C-terminal arginine residue.

In a typical peptide mass fingerprinting protocol, individual proteins in a sample are initially separated from others. A number of different separation methods are available (e.g. 1-dimensional or 2-dimensional, reverse-phase or normal-phase separation, by e.g. chromatography (including HPLC) or electrophoresis) and the separation may be based on any of a number of protein characteristics (e.g. isoelectric point, molecular weight, charge, hydrophobicity, etc.). Typically, 2D SDS-PAGE is used for peptide mass fingerprinting. 2D liquid chromatography (e.g. Multidimensional Protein Identification Technology, MudPIT) may also be used. The separation step can preferably interface directly with the mass spectrometer. One or more of the separated proteins are individually digested with a protease (typically trypsin) prior to mass spectrometry. The digestion step is commonly carried out in situ after separation (e.g. in a SDS-PAGE gel or chromatography medium) to facilitate extraction of the polypeptide from the separation medium (smaller digested fragments may diffuse out from the separation medium more readily). Accordingly, preparation of the mixture of peptides by digestion of a protein with a protease may be carried out in situ in the medium used for separation.

A peptide may be free in solution or, as an alternative, may be attached to a solid support, covalently or non-covalently. Where the peptide is attached to a solid support, it will be removed from the solid support for analysis by mass spectrometry.

In addition to the peptide(s), the sample may also include one or more solvents, one or more buffers, one or more salts, one or more detergents, one or more protease inhibitors, etc.

### Derivatisation with Label

Peptide(s) within a sample are reacted with a label to provide derivatised peptides for mass spectrometry. If the derivatised peptide contains an arginine residue then it can form both a stabilised ion species ([P]⁺) and a protonated ion molecular species ([P+H]⁺) that differ by one average mass unit. A preferred group of labels gives derivatised peptides that can form free radical ion species.

As well as providing a characteristic peak pattern for arginine-containing peptides, advantageous labels can improve the ionisation properties of the peptide. One such class of labels is trityl derivatives, as disclosed in European patent application 04104605.3 (copy enclosed). Preferred labels have formulae (IIa), (IIb) (IVai), (IVaii), (IVaiii), (IVbii), (IVbiii), (IVaiv) and (IVbiv), as defined in EP-04104605.3:

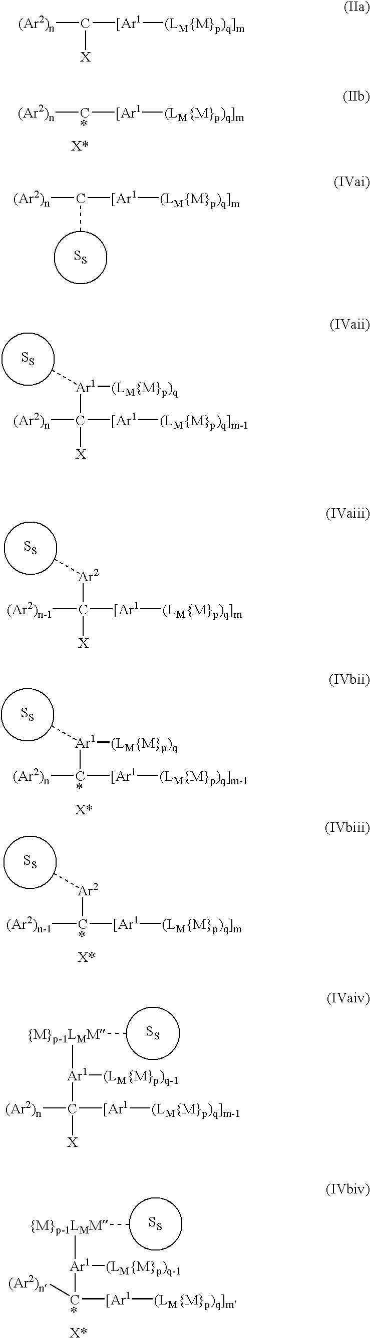

Preferred features of these formulae as disclosed in EP-04104605.3 are also preferred features of labels for use with this invention.

Particularly preferred labels are those which permit formation of ions during mass spectrometry that have a pKᵣ₊ value of at least zz, where zz is between −2 and +6. More preferably, zz is between −1 and +4.5. Most preferably, zz is between −1 and +0.5.

In order to react with a peptide, the label may be free in solution (e.g. the label can be added to the peptide and reacted in solution) or, as an alternative, may be attached (covalently or non-covalently) to a solid support (e.g. peptides can be added to immobilised label and subsequently released from the support for analysis by mass spectrometry).

The reaction may be carried out at any stage prior to analysis of the peptide(s) by mass spectrometry. For example, the reaction may be carried out before separation of the proteins in a sample. As an alternative, the reaction may be carried out following separation of the proteins in a sample but before digestion of one or more individual proteins. As a further alternative, the reaction may be carried out following digestion of a protein of interest to provide a mixture of peptides. Labelling before digestion gives fewer labels per original protein sequence than labelling after digestion.

For peptide mass fingerprinting, it is preferred that the peptides(s) in a peptide mixture are derivatised i.e. the reaction is preferably carried out following digestion of a protein of interest.

The derivatisation reaction may proceed directly or indirectly. For example, a group present on the peptide may react directly with a group on the label. Alternatively, the peptide may initially be derivatised with one or more suitable groups (e.g. N-hydroxysuccinimide) for subsequent reaction with a suitable group on the label. Thus, the present invention allows one or more steps of peptide manipulation prior to derivatisation with the label.

It is possible for a peptide to be labelled by two separate labels (e.g. one at the N-terminus and one on a lysine side chain). In such circumstances the inventors have observed only a singly-charged ion and so the only effect of double labelling is additional mass, rather than any change in charge characteristics. Thus peptides of the invention may carry one or more labels (e.g. 2, 3, 4, 5 or more).

The invention also provides a method of screening for labels that can react with a peptide to provide a derivatised peptide that, if the peptide contains an arginine residue, can form both a stabilised cation ion species ([P]⁺) and a protonated ion molecular species ([P+H]⁺) that differ by one average mass unit, comprising the steps:
a) obtaining a candidate label;
b) reacting the candidate label with an arginine-containing peptide to provide a derivatised arginine-containing peptide;
c) analysing the derivatised arginine-containing peptide by mass spectrometry to provide a mass spectrum; and
d) analysing the mass spectrum to determine if, after optional deisotoping, it contains a peak pattern for a peptide in which a first peak and a second peak are separated by one average mass unit.

The first peak in the spectrum analysed in step (d) may be (i) less abundant than the second peak, and (ii) of lower mass than the second peak.

If a spectrum contains the characteristic peak pattern (e.g. after deisotoping) then the candidate label is a label suitable for use with the invention.

Candidate labels may be derived from large libraries of synthetic or natural compounds. For instance, synthetic compound libraries are commercially available from MayBridge Chemical Co. (Revillet, Cornwall, UK) or Aldrich (Milwaukee, Wis.). Alternatively, libraries of natural compounds in the form of bacterial, fungal, plant and animal extracts may be used. Additionally, candidate labels may be synthetically produced using combinatorial chemistry either as individual compounds or as mixtures.

### Derivatised Peptides

The invention also provides a peptide with an N-terminal residue and including an arginine residue, characterised in that (a) a label is attached to the N-terminal residue of the peptide and (b) the peptide can form both a stabilised ion species ([P]⁺) and a protonated ion molecular species ([P+H]⁺) that differ by one average mass unit. These peptides are produced during the derivatisation of the peptides with suitable labels, as described above.

The peptide of the invention will typically also include further amino acids, each of which has a sidechain, and in some peptides of the invention a label may be attached to one or more of these sidechain(s), particularly to the sidechain of a lysine residue.

Preferably, the peptide comprises at least A amino acids, where A is 2 or more (e.g. 2, 3, 4, 5, 6, 7, 8, 9, 10, 11, 12, 13, 14, 15, 16, 17, 18, 19, 20, 21, 22, 23, 24, 25, 26, 27, 28, 29, or 30). Preferably, the peptide comprises at most B amino acids, where B is 100 or less (e.g. 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31 or 30).

The invention also provides ionic forms of such peptides, protonated ionic forms of such peptides, free radical forms of such peptides and free radical ionic forms of such peptides. Preferably, the ionic forms are cationic.

The invention also provides a mixture of these forms of the peptides of the invention. A mixture of these forms of the peptides of the invention includes 2 or more different peptides, e.g. >5 peptides, >10 peptides, >20 peptides, >30 peptides, >40 peptides, >50 peptides, >60 peptides, >70, peptides, >80 peptides, >90 peptides, >100 peptides, etc. The peptides in the mixture may each independently be present as an ionic form, a protonated ionic, a free radical form or a free radical ionic form.

The invention also provides a kit comprising: (a) a label for derivatisation of peptide(s) to provide derivatised peptides which, if the peptide contains an arginine residue, can form both a stabilised ion species ([P]+) and a protonated ion molecular species ([P+H]+) that differ by one average mass unit; and (b) one or more other components selected from the group consisting of: a separation medium (e.g. an electrophoresis gel or chromatography column), a protease, a protease inhibitor, a solvent, a buffer, a salt, a detergent, a mass standard and a matrix compound.

### Mass Spectrometry

Mass spectrometry of the derivatised peptide(s) will provide a mass spectrum. The mass spectrometer may comprise any of a number of combinations of ion source and mass analyser.

Suitable ion sources include, but are not limited to, matrix-assisted laser desorption ionisation (MALDI), fast atom bombardment (FAB) and electrospray ionisation (ESI) ion sources. Preferably, the ion source is a MALDI ion source. The MALDI ion source may be traditional MALDI source (under vacuum) or may be an atmospheric pressure MALDI (AP-MALDI) source.

Suitable mass analysers include, but are not limited to, time of flight (TOF), quadrupole time of flight (Q-TOF), ion trap (IT), quadrupole ion trap (Q-IT), triple quadrupole (QQQ) and Fourier transform ion cyclotron resonance (FTICR) mass analysers. Preferably, the mass analyser is a TOF mass analyser.

Preferably, the invention uses a MALDI-TOF mass spectrometer.

For MALDI-MS, a sample containing a peptide is mixed with a matrix compound prior to spotting onto a target plate. The matrix compound is selected such that it absorbs the wavelength of laser light which is to be used for ionisation, is able to co-crystallise with the peptide(s), is vacuum stable, causes desorption of the peptide(s) upon laser irradiation and promotes peptide ionisation. A wide variety of matrix compounds useful for peptides are known in the art, including alpha-cyano-4-hydroxycinnamic acid (CHCA), sinapic acid (SA), 2-(4-hydroxyphenylazo)benzoic acid (HABA), succinic acid, 2,6-dihydroxyacetophenone, ferulic acid, caffeic acid, glycerol and 4-nitroaniline.

The present invention therefore also provides a mixture of a derivatised peptide of the invention and a matrix compound.

As noted above, the reaction of a label with peptide(s) within a sample may be carried out at any stage prior to analysis of the peptide(s) by mass spectrometry. The reaction may be carried out after or, preferably, before mixing the peptide(s) with the matrix compound.

Mass spectrometry of the derivatised peptide(s) may include the analysis of mass standards added to the sample prior to mass spectrometry. Alternatively, one or more components already present in the sample may be used as a mass standard. For example, autoproteolytic fragments of a protease used to produce a peptide mixture are often used as mass standards.

Mass spectrometry of the derivatised peptide(s) may include more than one data collection step per sample. For example, tandem mass spectrometry may be used, in which the initial data collection step is followed by a second data collection step, as well known in the art (known as MS/MS). Where more than one data collection step per sample is employed, the mass analyser need not be the same for each data collection step, and further fragmentation of the ions may occur between data collection steps.

Preferably, the stabilised ion species ([P]⁺) and protonated ion molecular species ([P+H]⁺) are formed by loss of a hydroxyl group from the label during ionisation. Thus, the stabilised ion species ([P]⁺) is preferably [M−OH]⁺ and the protonated ion molecular species ([P+H]⁺) is preferably [M—OH+H]⁺ (where M represents the derivatised peptide molecule prior to ionisation).

### Analysis of Mass Spectrometry Data

A mass spectrum of a peptide may be analysed to identify if it contains a peak pattern for a peptide in which a first peak and a second peak are separated by one average mass unit. The first peak in the spectrum may be (i) less abundant, equally abundant, or more abundant than the second peak, and (ii) of lower mass than the second peak. Preferably, the first peak in the spectrum is (i) less abundant than the second peak, and (ii) of lower mass than the second peak.

The initial analysis of the raw mass spectrum of the peptide(s) may include deisotoping and/or identification of the monoisotopic masses of the peptide(s). The monoisotopic mass of a peptide is the mass of the lightest ion for that peptide (i.e. the mass of the ion that contains the lightest isotope of each of the elements that contribute to the isotopic distribution).

The initial analysis of the mass spectrum of the peptide(s) may also include the identification of the relative intensity of the peaks generated by each isotope.

There are a number of computer packages available for the automated analysis of mass spectra. Particularly, there are a number of computer packages available for the automated identification of monoisotopic masses of peptides from the mass spectrum and the intensities of the peaks within the isotopic distribution for each peptide.

The existence of an isotopic distribution in the mass spectrum of peptides is well known in the art. Modern mass spectrometers are capable of resolving the isotopic distribution of individual molecules, by separating ions containing ¹²C, ¹H and ¹⁶O from ions of the same molecule that contain one or more atoms of ¹³C, ²H or ¹⁷O. Thus, modern mass spectrometers are not limited to a determination of the average ion mass. Deisotoping of the mass spectrum is used to identify the monoisotopic mass for a peptide from the isotopic distribution pattern present in the mass spectrum. Various computer algorithms are known in the art for deisotoping mass spectra (e.g. ‘Collapse’, produced by Positive Probability Ltd). Deisotoping the mass spectrum is generally preceded by centroiding the peaks within each isotopic distribution to provide a number of defined peaks for each peptide. The pattern of centroided peaks is then deisotoped by comparison of the measured intensities of the peaks in each cluster against the intensities of peaks within generic template isotopic distributions for peptides.

Deisotoping is an easy way of revealing whether a set of peaks in a spectrum contains the pattern which is characteristic for Arg-containing peptides (e.g. see FIGS. 1 & 2). An alternative method involves a direct comparison of the actual isotope pattern with theoretical patterns (e.g. see FIGS. 1 and 3) without deisotoping.

The isotopic distribution for a peptide is dictated by the relative natural abundance of the isotopes of the elements present in the peptide, and all peptides normally display a similar isotopic distribution pattern. In contrast, the mass spectra of arginine-containing peptides derivatised with a suitable label are also influenced by the abundance of the protonated ion molecular species ([P+H]⁺). After deisotoping, derivatised peptides that contain an arginine residue will be represented by two peaks separated by one average mass unit. In contrast, derivatised peptides that do not contain an arginine residue will be represented by a single peak (e.g. see FIG. 2). Accordingly, the observation of the characteristic peak pattern indicates that the relevant peptide contains an arginine residue.

As described above, suitable labels give derivatised peptides that have the ability to form both a stabilised ion species ([P]⁺) and a protonated ion molecular species ([P+H]⁺) that differ by one average mass unit. Suitable labels may also give derivatised peptides that have the ability to form multiply charged ion species ([P]ⁿ⁺ and [P+H]ⁿ⁺, where n in an integer greater than 1) that differ by one average mass unit. Therefore, reference herein to the stabilised ion species ([P]⁺) and the protonated ion molecular species ([P+H]⁺) includes reference to multiply charged forms of those ion species.

It will be understood by those of skill in the art that for multiply charged ions the difference between the stabilised ion species and the protonated ion molecular species observed by mass spectrometry will be 1/n average mass unit (i.e. the difference will be determined by the number of charges on the ion). For example, for doubly charged ions ([p]²⁺ and [P+H]²⁺) the difference between the stabilised ion species and the protonated ion molecular species observed by mass spectrometry will be half an average mass unit. Therefore, reference herein to analysis of a mass spectrum to determine if it contains a peak pattern for a peptide in which a first monoisotopic mass and a second monoisotopic mass are separated by one average mass unit includes reference to analysis of a mass spectrum to determine if it contains a peak pattern for a peptide in which a first monoisotopic mass and a second monoisotopic mass are separated by a fraction of one average mass unit if multiply charged ions are observed.

The analysis of the mass spectrum may include the step of determining the ratio or relative abundance of modified and unmodified peptides (e.g. phosphorylated/unphosphorylated etc.).

The analysis of the mass spectrum may be carried out manually or may be automated. Preferably, the analysis of the mass spectrum is automated e.g. using a computer.

The present invention allows the improvement of computer packages for the automated identification of peptides that comprise arginine residues, via automated analysis of the peptide mass spectra.

### Database Searching

Database searching may be carried out using any suitable computer package.

A number of suitable computer packages for identifying molecules based on mass spectrometry fingerprints are available. Particularly, a number of suitable computer packages for identifying proteins based on mass spectrometry fingerprints are available, and these include PepSea, PeptIdent/MultIdent, MOWSE, MS-Fit (part of the ProteinProspector suite), PROWL, SEQUEST, MASCOT and ProFound.

These computer packages typically fall into three broad categories:
A. Algorithms that assign scores based on the number of experimental peptide masses that match peptide masses in the peptide database (e.g. PepSea, PeptIdent/MultIdent).
B. Algorithms that score matches based on the length of the peptide and protein (e.g. MOWSE, MS-Fit).
C. Algorithms that use probabilistic scoring methods to determine the significance of matches between experimental peptide masses and database peptide masses (e.g. PROWL, MASCOT).

The known computer packages can be improved by incorporation of the additional parameter of knowing whether or not the peptide comprises an arginine residue.

For example, the invention enables improved searching algorithms that filter false positive hits by discounting database peptides that either contain or lack an arginine residue, as appropriate. Search algorithms that incorporate discrimination based upon the additional parameter of whether or not the peptide comprises an arginine residue are predicted to provide greatly improved certainty for the search output. Alternatively, the invention enables simplification of the sequence space that needs to be searched for each peptide, by searching a database containing only sequences that either contain or lack an arginine residue, as appropriate e.g. double peaks can be searched against an Arg-containing database, whereas single peaks can be searched against an Arg-free database.

Preferably, the additional information provided by the present invention will be incorporated by the use of a database subset e.g. one which contains only Arg-containing peptide sequences and/or one which contains only Arg-free peptide sequences. The invention allows any sequence database to be split into (a) sequences that contain Arg and (b) sequences that do not contain Arg. A peak which is known to contain Arg by use of the invention can be searched against sub-database (a), while other peaks can be searched against sub-database (b), thereby greatly increasing efficiency.

In addition, the cleavage specificity of a protease may be incorporated in the search strategy, as is well known in the art. The combination of knowledge of the cleavage specificity of a protease and knowledge of the presence or absence of an arginine residue improves database searching accuracy by providing further structural constraints on the sequences.

In addition, the specificity of the chosen label may be incorporated in the search strategy. For example, the label may react only with certain sidechains present on the peptide, allowing identification of peptides comprising those sidechains (due to the presence of a peak in the mass spectrum for those peptides at a position governed by the mass of the label). This information is preferably combined with any other available structural constraints, such as the presence of an arginine residue or the cleavage specificity of a protease, to further improve searching accuracy.

The increase in certainty of the algorithm score provided by the methods of the present invention provides a significant improvement in peptide mass fingerprinting.

The invention provides a system for analysing a mass spectrum, comprising a module for:
a) receiving a mass spectrum; and
b) analysing the mass spectrum to determine if, after optional deisotoping, it contains a peak pattern for a peptide in which a first peak and a second peak are separated by one average mass unit.

The first peak in the spectrum analysed in step (b) may be (i) less abundant than the second peak, and (ii) of lower mass than the second peak.

The system of the invention may be a hardware system or a software system.

If the system is a hardware system, it may comprise a central processing unit; an input device for inputting requests; an output device; a memory; and at least one bus connecting the central processing unit, the memory, the input device and the output device. The memory should store the module, which is configured so that upon receiving a request to determine if a mass spectrum contains a peak pattern for a peptide in which a first peak and a second peak are separated by one average mass unit, it performs one or more steps for identification of that characteristic peak pattern. Thus, the invention provides a computer apparatus adapted to perform a method of the invention.

The invention also provides a computer program for analysing a mass spectrum, comprising a program module for:
a) receiving a mass spectrum; and
b) analysing the mass spectrum to determine if, after optional deisotoping, it contains a peak pattern for a peptide in which a first peak and a second peak are separated by one average mass unit.

The first peak in the spectrum analysed in step (b) may be (i) less abundant than the second peak, and (ii) of lower mass than the second peak.

The invention also provides a computer program product comprising a computer readable storage medium having stored thereon computer program means for receiving a mass spectrum and for analysing the mass spectrum to determine if, after optional deisotoping, the spectrum contains a peak pattern for a peptide in which a first peak and a second peak are separated by one average mass unit. Preferably, the first peak is less abundant than the second peak.

### BRIEF DESCRIPTION OF THE FIGURES

FIGS. 1 and 2 show a MALDI-TOF mass spectrum generated for a mixture of four peptides derived from a trypsin-digested polypeptide and derivatised according to the invention. FIG. 1 shows the “raw” spectrum and FIG. 2 shows the results of centroiding and deisotoping.

FIG. 3 shows the theoretical isotopic distribution for the LGEYGFQNALIVR peptide in its ionic ([P]⁺) and protonated ionic ([P+H]⁺) forms.

FIGS. 4 and 5 show the mass spectra of a BSA digest without (4) and with (5) labelling.

FIG. 6 shows a MALDI-TOF mass spectrum illustrating in-source fragmentation of a less stable label conjugated to GluFib B peptide.

FIG. 7 shows the MALDI-TOF mass spectra of guanidinated, unlabelled and dimethoxytrityl-labelled peptides derived from BSA.

FIG. 8 shows the MALDI-TOF mass spectrum for the H-ArgProLysPro-OH peptide.

FIG. 9 shows the effect of derivatisation of an arginine-containing peptide on the MALDI-TOF mass spectrum for that peptide.

FIG. 10 shows the MALDI-TOF mass spectra of a dimethoxytrityl-labelled (upper) and unlabelled (lower) myelin basic protein (MBP) tryptic digest.

### EXAMPLES

### Example 1

### Comparison of Isotopic Distributions for Specific Peptide(s)

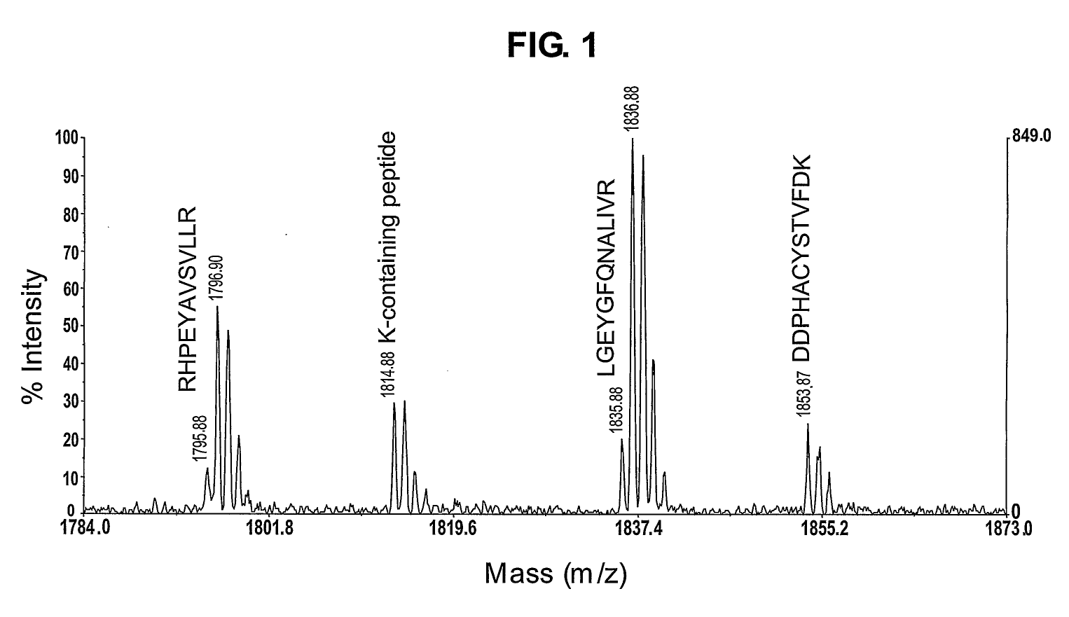

BSA was digested with trypsin and then derivatised with a dimethoxytrityl label of the invention. FIG. 1 shows a narrow portion of the mass spectrum generated by MALDI-TOF analysis of the digested protein. The characteristic peak pattern observed for the two peptides that contain arginine contrasts with the peak pattern observed for the two peptides that do not contain arginine residues. The peak pattern observed for the two peptides that do not contain arginine residues is the ‘traditional’ MALDI-TOF peak pattern for peptides.

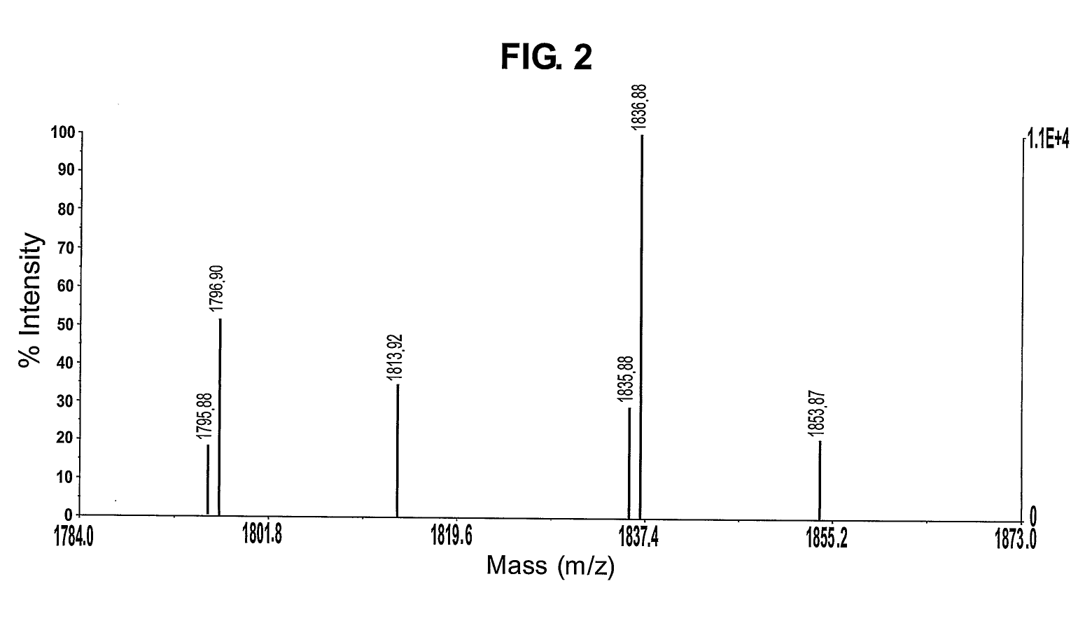

FIG. 2 shows the mass spectrum of FIG. 1 following centroiding and deisotoping. FIG. 2 shows that, after deisotoping, derivatised peptides that contain an arginine residue are represented by a peak pattern comprising a first peak and a second peak separated by one average mass unit. In this example, the first peak is less abundant than the second peak. In contrast, derivatised peptides that do not contain an arginine residue are represented by a single peak.

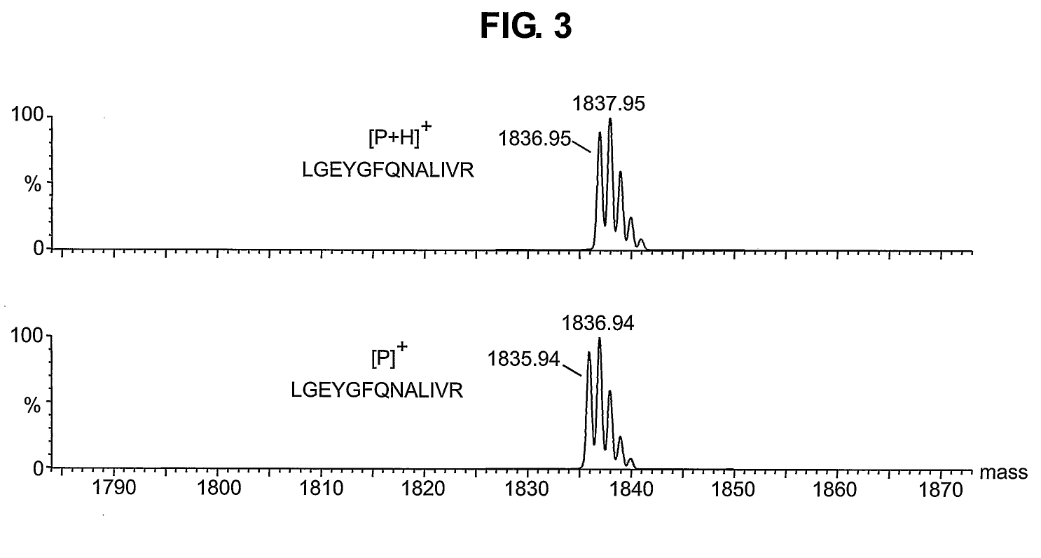

FIG. 3 shows the isotopic distribution peaks which would be expected for the LGEYGFQNALIVR fragment. A comparison of these theoretical patterns with the actual pattern seen in FIG. 1 reveals the effect of the invention.

The observed difference in the isotopic distribution for each peptide, and the consequent difference in the peaks present in the deisotoped spectrum, enables the discrimination of arginine-containing peptides from other peptides in the mass spectrum.

### Example 2

### BSA Fragmentation and Mass Spectrometry

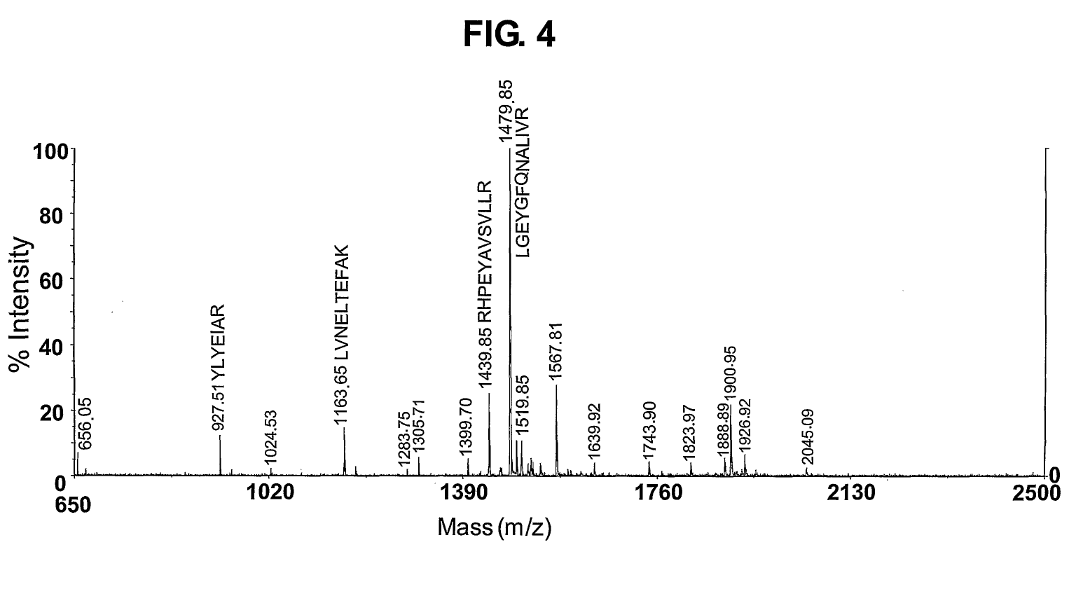

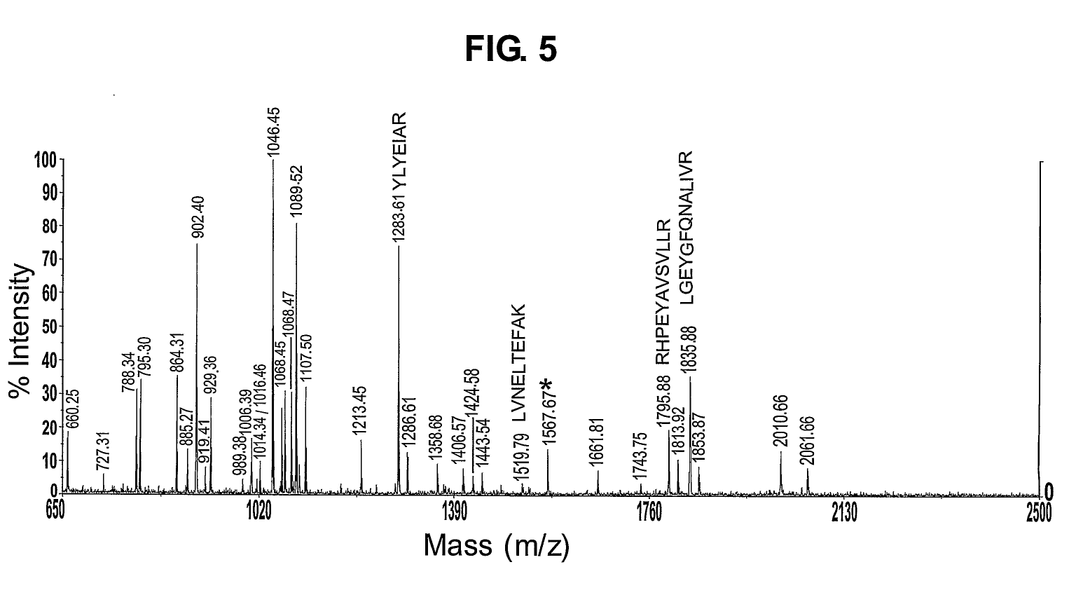

Bovine serum albumin (BSA) was digested with trypsin and analysed by MALDI-TOF mass spectrometry. The resulting spectrum is shown in FIG. 4. The experiment was repeated, but the peptide mixture was labelled with a dimethoxytrityl label after trypsin digestion. The spectrum in FIG. 5 shows the dramatic increase in visible ions due to the label. Four specific peptides have been highlighted in both spectra.

### Example 3

### Improvement in Peptide Mass Fingerprinting

Three proteins (BSA, β-casein and ADH) were digested with trypsin and the resulting peptides analysed by MALDI-TOF mass spectrometry with or without derivatisation. The number of peptides identified for each protein is shown below. The theoretical total number of peptides that would be produced by trypsin digestion of each protein was calculated in silico and is shown in the second column of the table:

| | | Total number of | | | |
|---|---|---|---|---|---|
| | Number of | peptides identified | | MASCOT search score* | |
| Protein | theoretical peptides⁺ | Underivatised | Derivatised | Underivatised | Derivatised |
| BSA | 144 | 14 (10%) | 41 (28%) | 132 | 126 |
| β-casein | 27 | 4 (15%) | 13 (48%) | no match | 123 |
| ADH | 60 | 7 (12%) | 18 (30%) | 77 | 111 |

⁺The number of theoretical peptides for each protein was generated assuming one missed cleavage and disregarding di- and mono-amino acids generated.
*Score is −10 * Log(P), where P is the probability that the observed match is a random event. Protein scores greater than 63 are significant (p < 0.05).

Derivatisation of peptides with trityl groups of the invention thus improves detection, as a significantly larger number of peptides was detected for each of the three proteins when derivatisation was used. Furthermore, protein identification by mass fingerprinting can be improved.

Taking β-casein as an example, the number of detectable fragments more than tripled, and the derivatised spectrum allowed a MASCOT-based identification which was not previously possible.

For BSA, the confidence of the MASCOT prediction was not significantly altered. However, derivatisation of the peptides with a substituted dimethoxytrityl group results in an increase in the mass of the observed peptides of around 300 mass units (the mass of the trityl residue). As a result, larger peptides no longer fell within the range of the mass spectrometer, and shorter peptides were observed. The shift to shorter peptides decreases the sequence certainty that can be assigned to each peptide because the probability of a random match is higher. Accordingly, it would be expected that derivatisation of the peptides would result in significantly lower scores after database searching, which explains the slight decrease in the BSA score. On the other hand, the large increase in the number of peptides detected for BSA was sufficient to overcome this effect. Furthermore, the large increase in the number of peptides detected for ADH and β-casein enabled a significant increase in the scores for those proteins, despite the expected decrease.

Database searching with the additional parameter of whether the terminating amino acid was a lysine or arginine has been predicted to increase the certainty of a score by at least ten-fold (in addition to the effect on the score of the increased number of peptides detected).

Database searching for other protease digests (i.e. those that may produce peptides with C-termini other than Lys or Arg) with the additional parameter of whether the peptide comprises an arginine residue has been predicted to increase the certainty of a score by at least 10% (in addition to the effect on the score of the increased number of peptides detected).

### Example 4

### Confirmation of Direct Effect of Label on Characteristic Peak Pattern

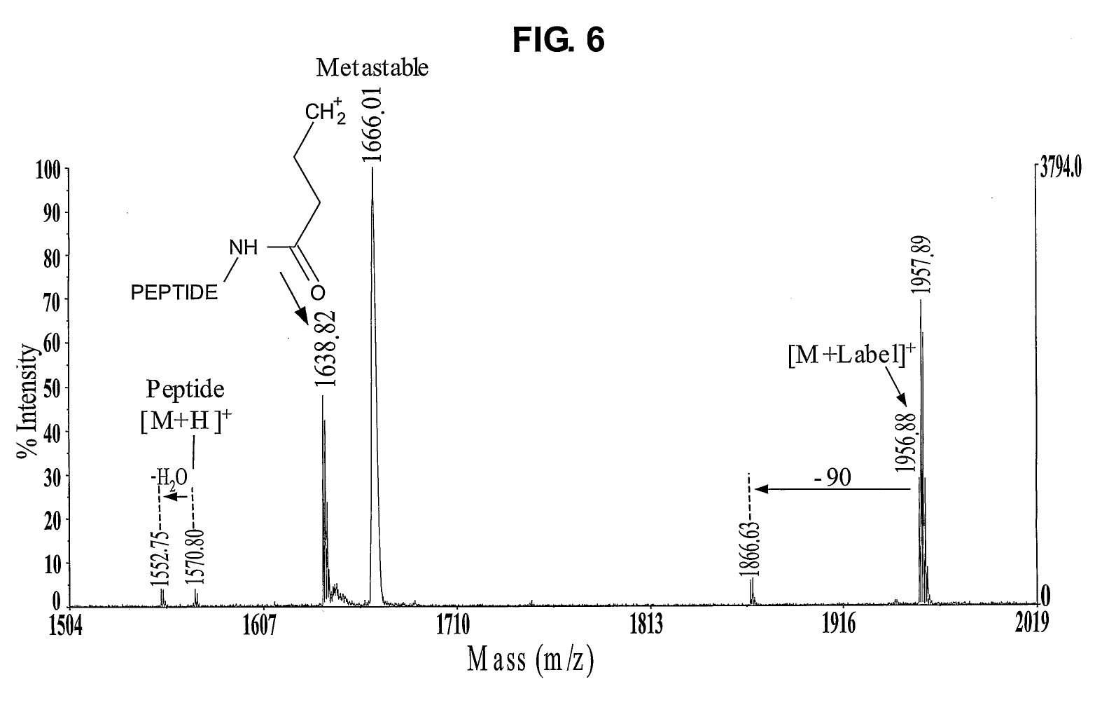

To confirm that the characteristic peak pattern is the direct result of the derivatisation of the peptide, a less stable label containing an ester linkage between the trityl moiety and the peptide was used. Cleavage of that label from a derivatised GluFib B peptide by MALDI-TOF-ISD-MS shows a fragment ion (at m/z 1638.8) that has the ‘traditional’ peptide isotope distribution pattern, whereas the molecular ion (to which the label is attached) has the characteristic peak pattern of the invention (see FIG. 6). The results shown in FIG. 6 confirm that the trityl moiety is responsible for the characteristic peak pattern observed for arginine-containing peptides.

### Example 5

### The Effect of Peptide Sequence and Number of Labels

The position of the arginine residue and the number of labels attached to the peptide does not affect the formation of the characteristic peak pattern, as highlighted by the data below.

| Bradykinin, Substance P and GluFib B Peptides | | | Peptide + label | | Isotope |
|---|---|---|---|---|---|
| | Conc. | Calc. m/z | | Obs. | Unusual |
| Peptide | nmol/μl | peptide | Calc. m/z^{a} | m/z^{b} | pattern? |
| H-Arg-Pro-Pro-GlyPheSerProPheArg-OH | 0.94 | 1060.56 | 1416.71 | 1416.64 | ✓ |
| H-ArgProProGlyPhe-OH | 1.09 | 573.30 | 929.45 | 929.17 | ✓ |
| H-ArgProProGlyPheSerPro-OH | 1.13 | 757.39 | 1113.54 | 1113.24 | ✓ |
| H-ProProGlyPheSerPro-OH | 1.66 | 601.29 | 957.43 | 957.25 | X |
| H-ProProGlyPheSerProPheArg-OH | 0.88 | 904.46 | 1260.60 | 1260.25 | ✓ |
| H-ArgProLysProGlnGlnPhePheGlyLeuMet-NH₂ | 1.00 | 1347.72 | 1703.87 | 1703.15 | ✓ |
| H-ArgProLysPro-OH | 0.70 | 497.31 | 1209.62* | 1209.11* | ✓ |
| H-ArgProLysProGlnGlnPhePheGly-OH | 0.52 | 1103.60 | 1816.90* | 1817.29* | ✓ |
| H-ProGlnGlnPhePheGlyLeuMet(O)—NH₂ | 0.93 | 981.47 | 1338.62 | 1338.27 | X |
| H-GluGlyValAsnAspAsnGluGluGlyPhePheSerAlaArg-OH | 0.63 | 1570.66 | 1926.81 | 1926.20 | ✓ |

^{a}Calculated monoisotopic mass of [M + Label]+
^{b}Observed monoisotopic mass of [M + Label]+
*Calculated monoisotopic mass of di-substituted peptide [M + 2Labels]+

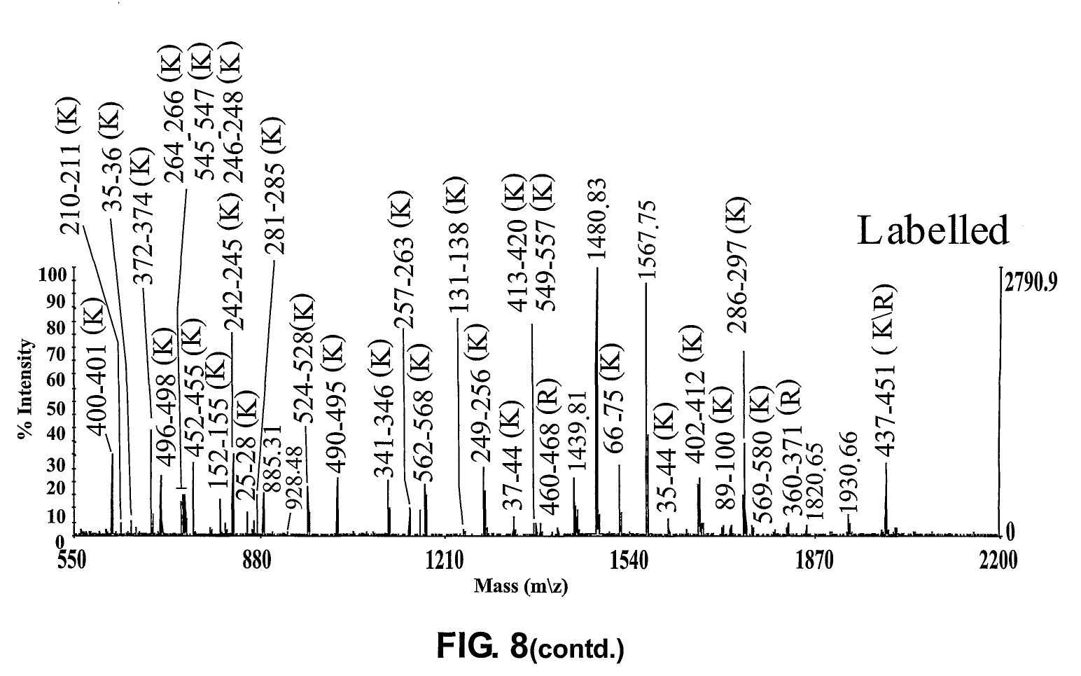

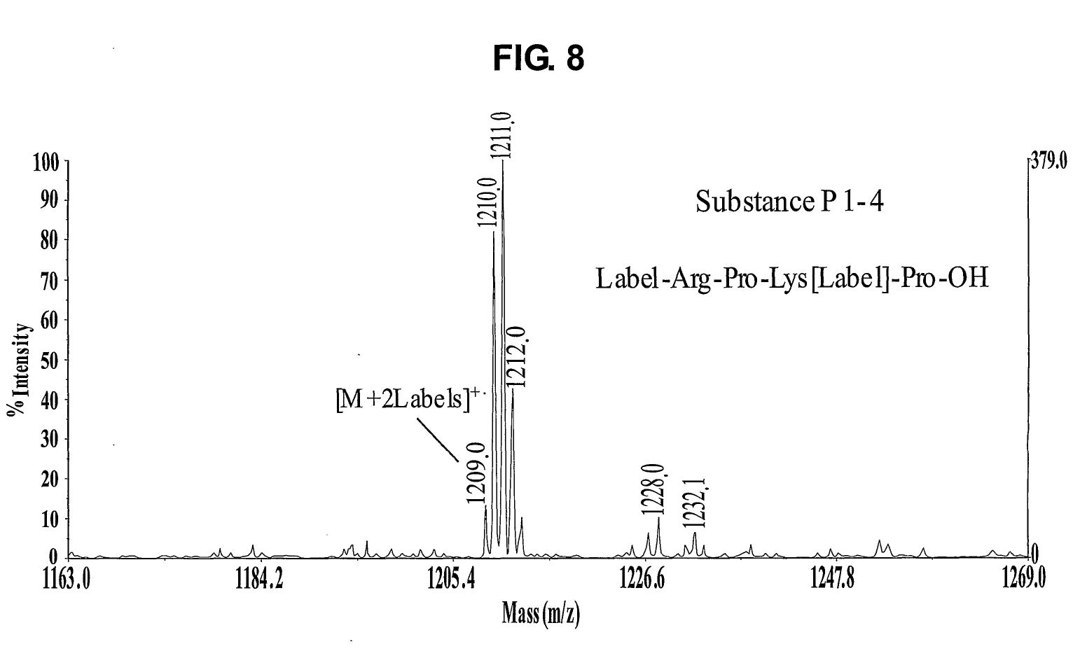

The MALDI-TOF mass spectrum for the H-ArgProLysPro-OH peptide, labelled at both its N-terminal Arg residue and at the internal Lys residue, is provided in FIG. 8. FIG. 8 confirms that the characteristic peak pattern is observed even when two labels are attached to a short peptide.

### Example 6

### Modulation of the Characteristic Peak Pattern

As described herein, the characteristic peak pattern of the invention is observed following formation of two molecular species, a stabilised ion species ([P]⁺) and a protonated ion molecular species ([P+H]⁺), that differ by one average mass unit. The stabilised ion species ([P]⁺) may be less abundant than the protonated ion molecular species ([P+H]⁺), but may also be more abundant or equally abundant.

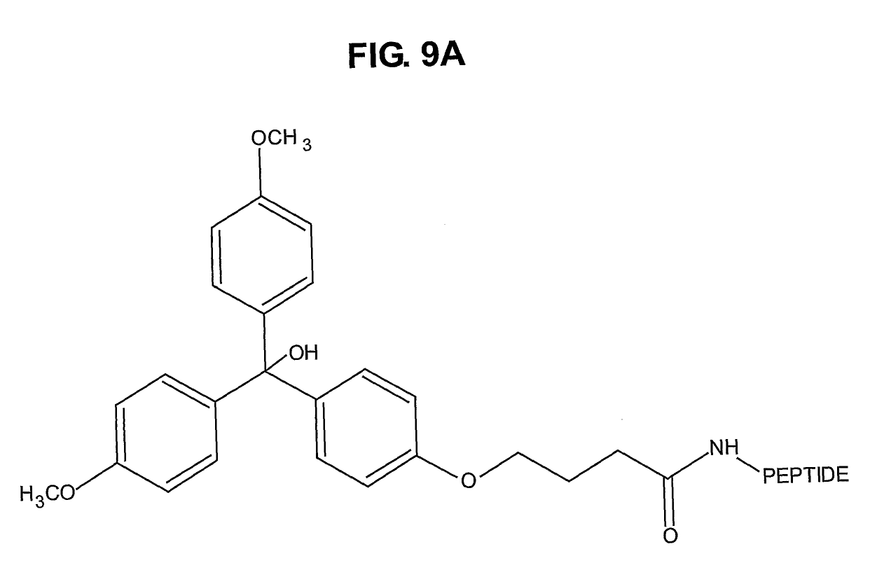

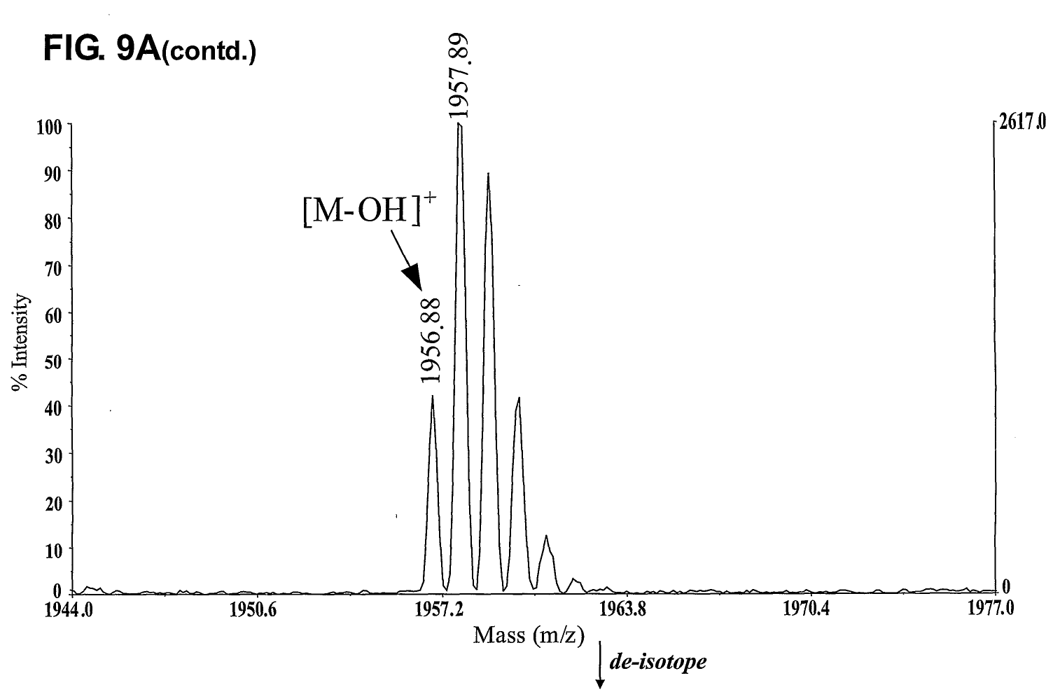

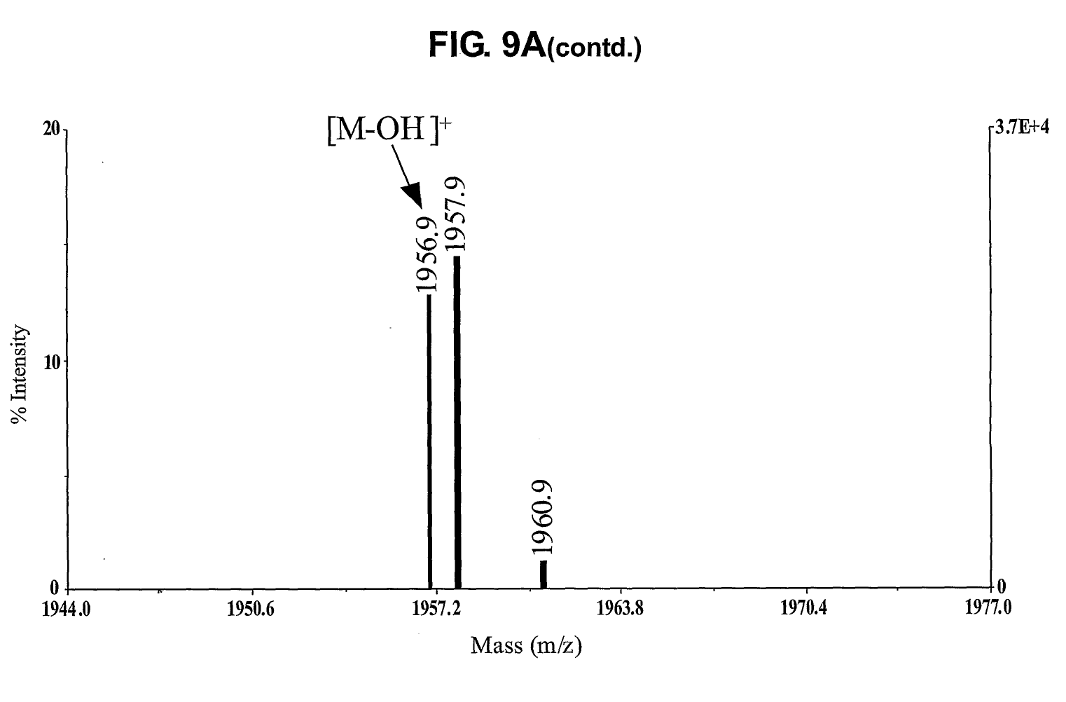

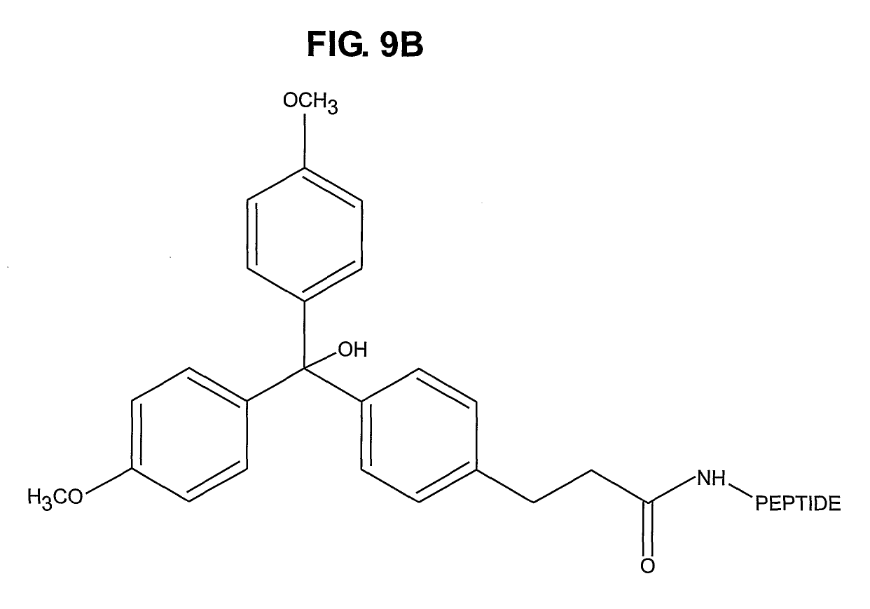

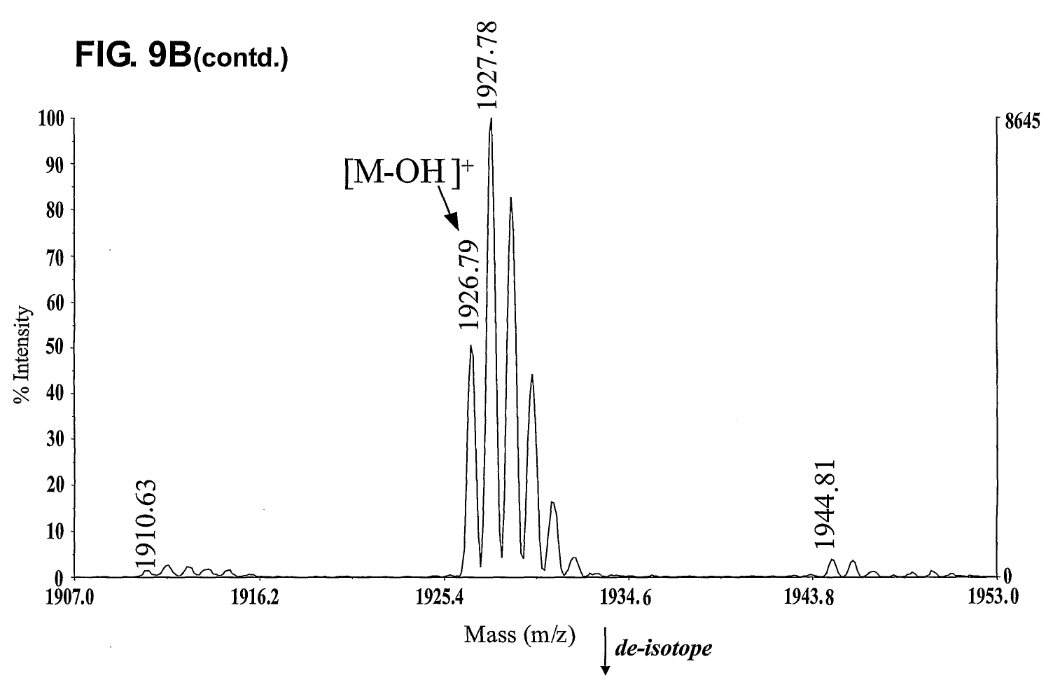

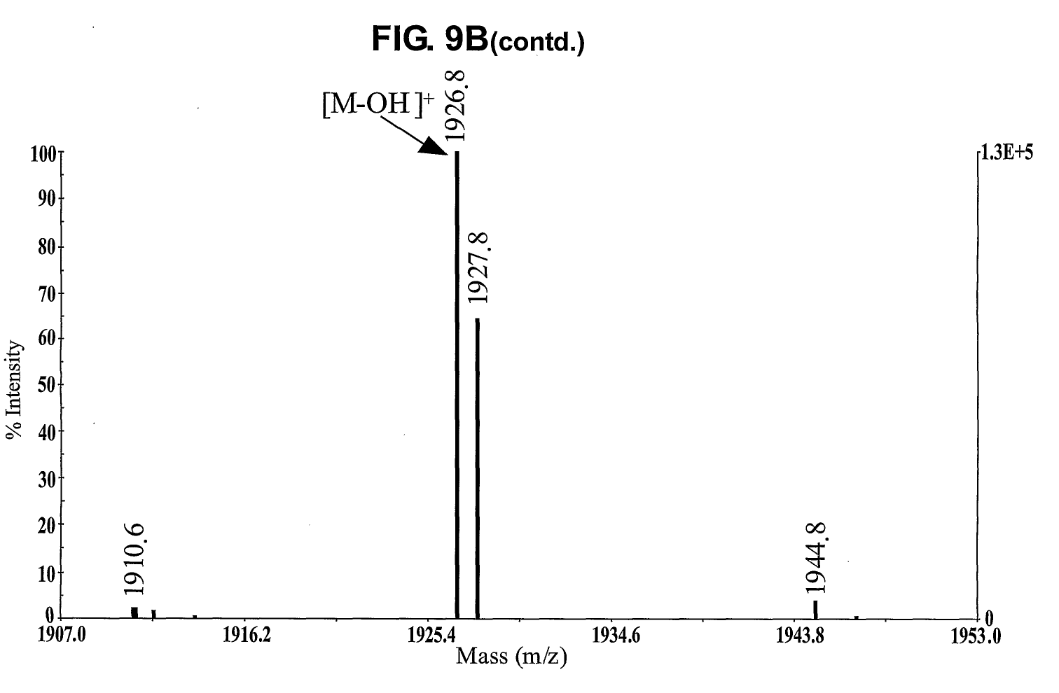

The specific chemical entity chosen for labelling the peptides affects the relative abundance of the stabilised ion species ([P]⁺) and the protonated ion molecular species ([P+H]⁺), as illustrated in FIG. 9. Both of the spectra in FIG. 9 include the characteristic peak pattern of the invention, comprising a first peak and a second peak separated by one average mass unit.

### Example 7

### Analysis of Post-Translationally Modified Proteins

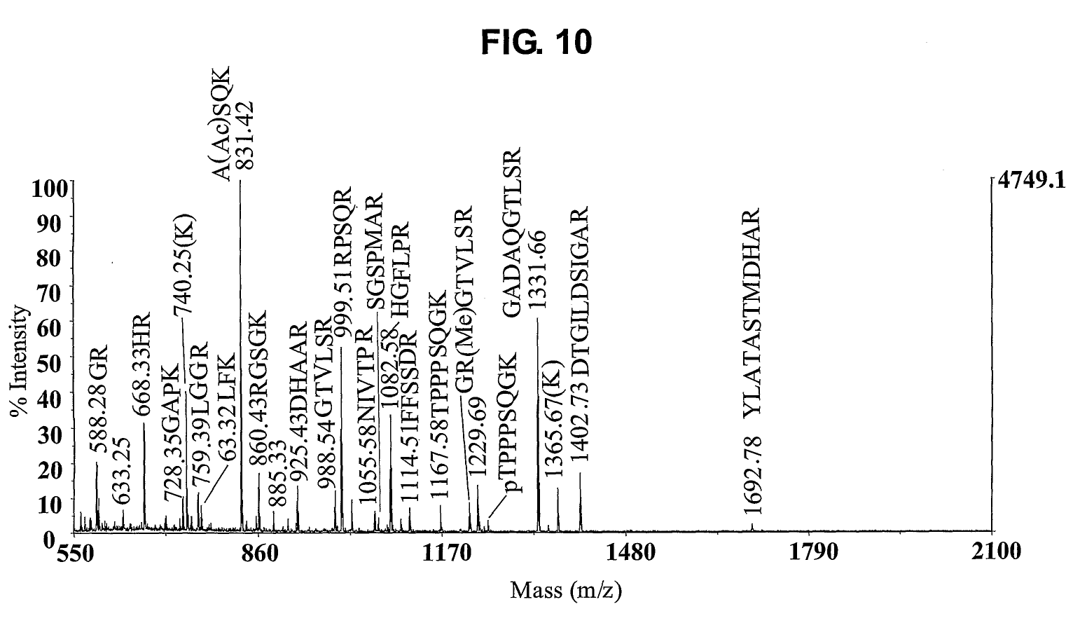

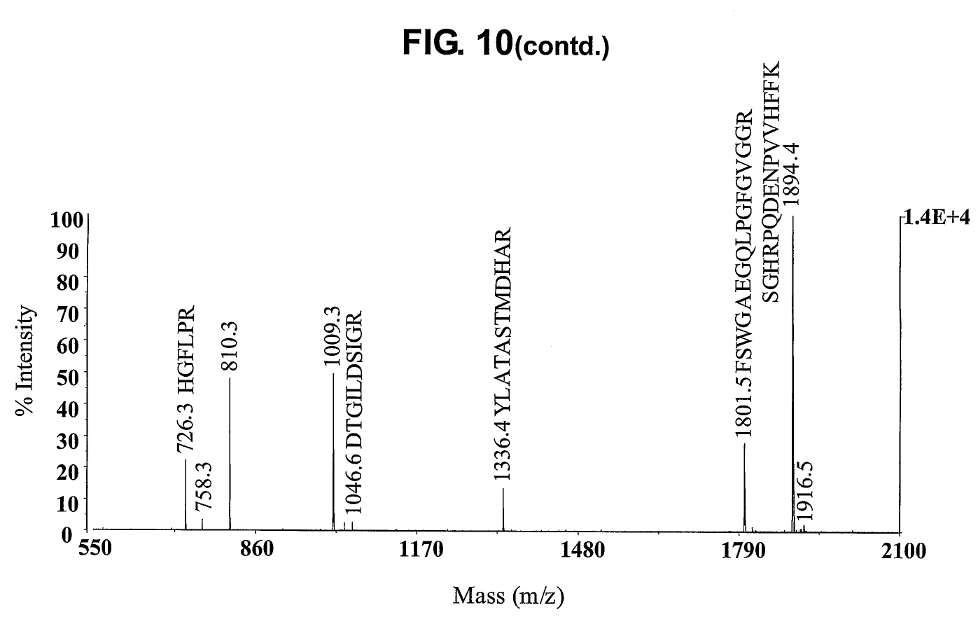

FIG. 10 shows the comparison of the MALDI-TOF mass spectra obtained for dimethoxytrityl-labelled and unlabelled peptides derived from a post-translationally modified protein, myelin basic protein (MBP). Three peptides present in the mass spectrum for the labelled peptides were found to contain the following modifications; an acetylated C-terminus (m/z 831.4), a methylated arginine (m/z 1215.7) and a phosphorylated threonine (m/z 1247.7). None of those modifications were identifiable from the mass spectrum for the unlabelled peptides. The ion at m/z 1215.7 shared the same molecular weight and isotope patterning as the di-dimethoxytritylated peptide, RGSGK but there was no evidence of this amino acid composition within the MALDI-CID-QTOF-MS/MS spectrum. The presence of the methylated arginine and its position within the amino acid sequence was confirmed using tandem mass spectrometry.

Notably, the mass spectrum shown in FIG. 10 includes an unphosphorylated peptide (m/z 1167.6), with no indication of a metastable ion that would denote a fragmentation process from the molecular ion of the phosphopeptide. Thus, the mass spectrum shown in FIG. 10 illustrates the possibility of determining the relative abundance of the phosphorylated and unphosphorylated form of a peptide from a single mass spectrum.

The same experiment was performed with a ‘cooler’ neutral pH matrix to reduce in-source fragmentation of the phosphate group within the mass spectrometer in order to check if the unlabelled peptide digest could produce a similar effect. In that experiment, no predicted phosphopeptides of the trypsin digest were detected, although the number of peptides increased from 5 to 9 as compared to the mass spectrum obtained when using α-cyano-4-hydroxycinnamic acid matrix.

The identification of phosphopeptides within a MALDI-TOF mass spectrum when a α-cyano-4-hydroxycinnamic acid matrix is used is of interest. To investigate further a labelled peptide containing a phosphorylated threonine residue was analysed by MALDI-TOF-MS. The mass spectrum showed good signal intensity at the 294 fmol/spot level and a further possible phosphorylated peptide with an additional asparagine residue was identified. In comparison, the underivatized peptide analysed using the same matrix required 400 fmol/spot for a 6-fold decrease in signal intensity with no identification of the additional product.

Nano-LC-QTOF-MS/MS experiments are commonly used to determine protein identity from a trypsin digest mixture when MALDI-TOF mass spectrometry fails to produce a confident database search result. Dimethoxytrityl-labelled MBP tryptic peptides were used to perform such an experiment to look at the compatibility of the labelling mixture with nLC-QTOF-MS/MS procedures. The results showed only two unlabelled peptides; one from MBP (DTGILDSIGR) and the other from trypsin related peptide (VATVSLPR). The majority of the peptides correlated with those found in the labelled MALDI-TOF mass spectrum and their amino sequences where confirmed from the MS/MS spectra. Furthermore, it appeared that many of the lysine-containing peptides carried the dimethoxytrityl label on the lysine side chain rather than the N-terminus.

### Example 8

### Analysis of Peptides Containing Homo-Arginine Residues

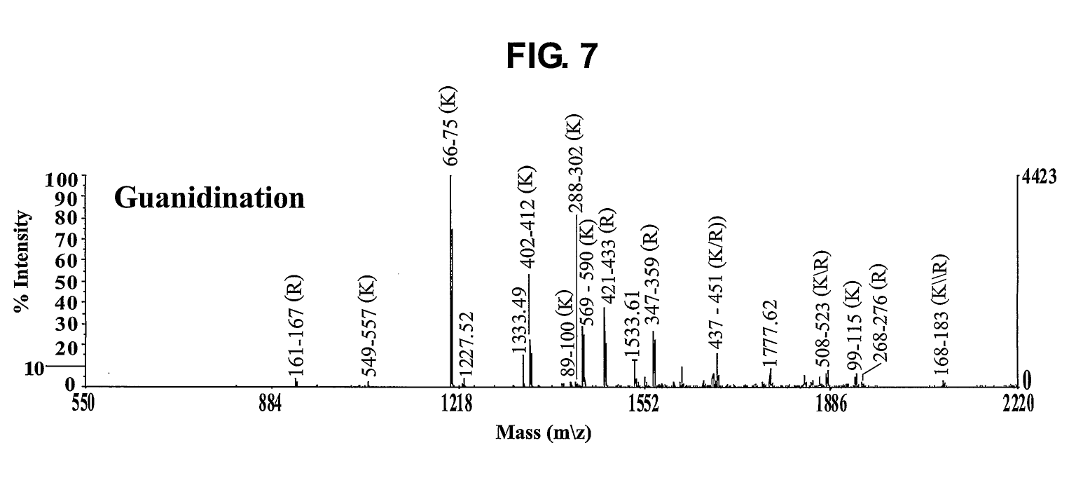

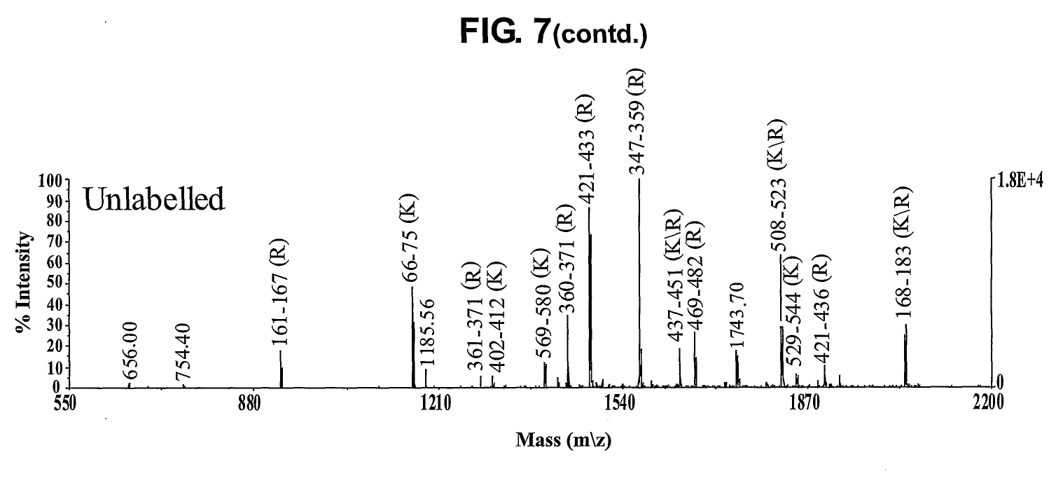

Conversion of a lysine residue to homo-arginine via guanidination has previously been shown to increase the ionisation of lysine-containing peptides and, when compared with the underivatized mass spectrum, allows the lysine-containing peptides to be identified by their mass shift. Tryptic digests of MBP, BSA and ADH were sequentially guanidinated and labelled with dimethoxytrityl to determine (a) if the peptide coverage increased and (b) if the arginine patterning effect after labelling would occur for homo-arginine-containing peptides. FIG. 7 shows the MALDI-TOF mass spectra of guanidinated, unlabelled and dimethoxytrityl-labelled peptides derived from BSA. The highest number of BSA peptides was observed for the dimethoxytrityl-labelled peptides. The unlabelled and guanidinated peptides showed the same number of peptides identified, both with different sequences. The homo-arginine-containing peptides, from the guanidination reaction, when labelled with dimethoxytrityl showed the same characteristic peak pattern as those peptides containing arginine. Thus, the spectra shown in FIG. 7 confirm the conclusion that the characteristic peak pattern is caused by protonation of a basic amino acid residue. The guanidination of peptides did not increase the peptide coverage of the chosen protein digests when compared to labelled peptides.

It will be understood that the invention is described above by way of example only and modifications may be made whilst remaining within the scope and spirit of the invention.

1. A method of analysing a peptide by mass spectrometry, comprising the steps of: (a) reacting the peptide with a label to provide a derivatised peptide that, if the peptide contains an arginine residue, can form both a stabilised ion species ([P]+) and a protonated ion molecular species ([P+H]+) that differ by one average mass unit; and (b) analysing the derivatised peptide by mass spectrometry to provide a mass spectrum.

2. The method of claim 1, further comprising the step of: (c) analysing the mass spectrum to determine if it contains a peak pattern for a peptide in which a first monoisotopic mass peak and a second monoisotopic mass peak are separated by one average mass unit and in which the first peak is of lower mass than the second peak and is less abundant than the second peak.

3. A method of identifying a protein by mass spectrometry, comprising the steps of: (a) obtaining a mass spectrum of a mixture of peptides derived from a protein, wherein the peptides are derivatised with a label that provides derivatised peptides that, if the peptide contains an arginine residue, can form both a stabilised ion species ([P]+) and a protonated ion molecular species ([P+H]+) that differ by one average mass unit; (b) analysing the mass spectrum to identify if, after optional deisotoping, it contains a peak pattern for a peptide in which a first peak and a second peak are separated by one average mass unit and in which the first peak is less abundant than the second peak; and (c) searching a database using information generated in step (b) to identify the protein.

4. The method of claim 3, wherein step (b) includes identifying monoisotopic masses of the peptides, and step (c) uses the monoisotopic masses.

5. The method of any preceding claim, wherein the peptide is a proteolytic fragment.

6. The method of claim 5, wherein the peptide is a trypsin fragment.

7. The method of any preceding claim, wherein the derivatised peptide can form free radical ions.

8. The method of any preceding claim, wherein the label enhances the ionisation properties of the peptide relative to non-derivatised peptide.

9. The method of any preceding claim, wherein the label has formula (IIa), (IIb) (IVai), (IVaii), (IVaiii), (IVbii), (IVbiii), (IVaiv) and (IVbiv) described herein.

10. The method of any preceding claim, wherein the mass spectrometry uses a MALDI source.

11. The method of any preceding claim, wherein the mass spectrometry uses a TOF mass analyser.

12. The method of any preceding claim, wherein mass spectrometry output data is used to identify an amino acid sequence.

13. The method of claim 12, wherein the presence or absence of an arginine residue in a peak on a mass spectrum is used to reduce the number of possible identified amino acid sequences.

14. The method of any preceding claim, wherein the peptide is phosphorylated.

15. A peptide with an N-terminal residue and including an arginine residue, characterised in that (a) a label is attached to the N-terminal residue of the peptide and (b) the peptide can form both a stabilised ion species ([P]⁺) and a protonated ion molecular species ([P+H]⁺) that differ by one average mass unit.

16. The peptide of claim 15, comprising at least 5 amino acids.

17. The peptide of claim 15 or claim 16, in ionic and/or free radical form.

18. The peptide of any one of claims 15 to 17, wherein the peptide is phosphorylated.

19. A kit comprising: (a) a label for derivatisation of peptide(s) to provide derivatised peptides which, if the peptide contains an arginine residue, can form both a stabilised ion species ([P]+) and a protonated ion molecular species ([P+H]+) that differ by one average mass unit; and (b) one or more other components selected from the group consisting of: a separation medium, a protease, a protease inhibitor, a solvent, a buffer, a salt, a detergent, a mass standard and a matrix compound.

20. A system for analysing a mass spectrum, comprising a module for: (a) receiving a mass spectrum; and (b) analysing the mass spectrum to determine if, after optional deisotoping, it contains a peak pattern for a peptide in which a first peak and a second peak are separated by one average mass unit and in which the first peak is less abundant than the second peak.

21. The system of claim 20, which is a hardware system or a software system.

22. A computer program for analysing a mass spectrum, comprising a program module for: (a) receiving a mass spectrum; and (b) analysing the mass spectrum to determine if, after optional deisotoping, it contains a peak pattern for a peptide in which a first peak and a second peak are separated by one average mass unit and in which the first peak is less abundant than the second peak.

23. A method of analysing a deisotoped peptide mass spectrum, comprising the step of analysing the spectrum to determine if it contains a peak pattern for a peptide in which a first peak and a second peak are separated by one average mass unit and in which the first peak is less abundant than the second peak and has a lower mass than the second peak.

24. A method of screening for labels that can react with a peptide to provide a derivatised peptide that, if the peptide contains an arginine residue, can form both a stabilised cation ion species ([P]+) and a protonated ion molecular species ([P+H]+) that differ by one average mass unit, comprising the steps of: (a) obtaining a candidate label; (b) reacting the candidate label with an arginine-containing peptide to provide a derivatised arginine-containing peptide; (c) analysing the derivatised arginine-containing peptide by mass spectrometry to provide a mass spectrum; (d) analysing the mass spectrum to determine if, after deisotoping, it contains a peak pattern for a peptide in which a first peak and a second peak are separated by one average mass unit and in which the first peak is less abundant than and has lower mass than the second peak.
